(19) United States
(12) Patent Application Publication
(10) Pub. No.: US 2025/0349384 A1
(43) Pub. Date: Nov. 13, 2025

(54) AUTOMATED IDENTIFICATION OF GENES ASSOCIATED WITH PHENOTYPES

(71) Applicant: University of Utah Research Foundation, Salt Lake City, UT (US)

(72) Inventors: Brendan Daniel O'Fallon, Salt Lake City, UT (US); Trisha Gulati, Salt Lake City, UT (US); Hunter Best, Salt Lake City, UT (US)

(21) Appl. No.: 19/063,034

(22) Filed: Feb. 25, 2025

Related U.S. Application Data
(60) Provisional application No. 63/644,225, filed on May 8, 2024.

(51) Int. Cl. G16B 20/00; G16B 40/20

(52) U.S. Cl. CPC G16B 20/00; G16B 40/20

(57) ABSTRACT

The present disclosure relates to systems and methods for identifying genes associated with phenotypes. A list of phenotypes is provided as input and the systems and methods automatically provide an output with a list of genes associated with the phenotypes provided. The systems and method analyze assertions linking a gene to a phenotype using a graph-based algorithm to identify the genes associated with the phenotypes.

### CROSS-REFERENCE TO RELATED APPLICATIONS

This application claims priority to and the benefit of U.S. Provisional Patent Application No. 63/644,225, filed May 8, 2024, which is incorporated herein by reference in its entirety.

### BACKGROUND

Phenotype Driven Analysis (PDA) is frequently used when identifying clinically significant variants (mutations) in an exome or genome sample. PDA facilitates clinical interpretation of exome and genome cases by identifying genomic features such as protein-coding genes, non-coding ribonucleic acid (ncRNAs), or other regions, that are associated with the clinical features observed in a specific patient.

### BRIEF SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

Some implementations relate to a method. The method includes receiving an input with a plurality of phenotypes. The method includes analyzing assertions using a graph-based algorithm to determine genes associated with the plurality of phenotypes, wherein each assertion is in a standard format that associates a gene with a phenotype. The method includes outputting a gene list with the genes associated with the plurality of phenotypes.

Some implementations relate to a system. The system includes a memory to store data and instructions; and a processor operable to communicate with the memory, wherein the processor is operable to: receive an input with a plurality of phenotypes; analyze assertions using a graph-based algorithm to determine genes associated with the plurality of phenotypes, wherein each assertion is in a standard format that associates a gene with a phenotype; and output a gene list with the genes associated with the plurality of phenotypes.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 illustrates an example environment for identifying genes associated with phenotypes in accordance with implementations of the present disclosure.

FIG. 2 illustrates an example phenotype to gene assertions for use with implementations of the present disclosure.

FIG. 3 illustrates an example output of genes in accordance with implementations of the present disclosure.

FIG. 4 illustrates an example graph for use with an algorithm in accordance with implementations of the present disclosure.

FIG. 5 illustrates a graph with reported pathogenic genes from clinical exome probands in accordance with implementations of the present disclosure.

FIG. 6 illustrates a graph with reported pathogenic genes from clinical exome trios in accordance with implementations of the present disclosure.

FIG. 7 illustrates a method for identifying genes associated with phenotypes in accordance with implementations of the present disclosure.

FIG. 8 illustrates components that may be included within a computer system.

### DETAILED DESCRIPTION

This disclosure generally relates to identifying genes associated with phenotypes. Phenotype Driven Analysis (PDA) is frequently used when identifying clinically significant variants (mutations) in an exome or genome sample. Phenotypes are observable traits or characteristics of an organism. Examples of phenotypes include height, eye color, blood type, hearing loss, seizures, or telangiectasias. An individual's phenotype is determined by both genomic makeup (genotype) and environmental factors. In clinical genomics, the Human Phenotype Ontology (HPO) provides a curated set of all known phenotypes.

The present disclosure provides systems and methods that facilitates the identification of genes that have a clinical association with one or more phenotypes. A list of phenotypes are provided as input and the systems and methods of the present disclosure automatically provide an output with a list of genes likely associated with the phenotypes provided. The present disclosure includes a number of practical applications that provide benefits and/or solve problems associated with automated identification of genes associated with phenotypes. Examples of these applications and benefits are discussed in further detail below.

The systems and methods of the present disclosure converts information from a variety of sources into a standard format that contains gene-to-phenotype associations. The standard format creates an extensible and flexible interface that accommodates multiple sources of information and allows additional sources to be added easily to the systems. The systems and methods use assertions linking a gene to a phenotype to identify the genes associated with a list of phenotypes. In some implementations, the systems and methods use a graph-based algorithm for scoring genes and their relevance to a given list of phenotypes.

The systems and methods provide a clinical decision tool that receives an input with phenotypes and provides an output with a list of genes likely associated with the phenotypes provided as inputs. The clinical decision tool uses the assertions linking a gene to a phenotype to identify the genes likely associated with the phenotypes. For example, users input a list of phenotypes (typically describing an individual with a suspected genetic disorder) and the clinical decision support tool outputs genes likely to be associated with the phenotypes provided as input. In some implementations, the clinical decision tool uses a graph-based algorithm for scoring genes and a relevance of genes to a given list of phenotypes. The output of the clinical decision tool includes a list of genes identified as likely associated with the phenotypes inputted. In some implementations, the output includes an overall gene relevance score to the phenotypes provided in the phenotype input. In some implementations, the output includes for each phenotype an individual score indicating a relevance of a gene to each phenotype. In some implementations, the output includes the source where the information was pulled for the assertions and the scores.

The clinical decision tool may be used as a discovery tool to identify different genes associated with phenotypes. In addition, the clinical decision tool may be used to aid users in finding pathogenic variants faster.

One technical advantage of the systems and methods of the present disclosure is providing fast and accurate results. The graph-based algorithm takes only a few seconds to score the full set of human genes, facilitating rapid turn-around for clinical genome cases. Another technical advantage of the systems and methods of the present disclosure is creating a standardized format of assertions linking a gene to phenotypes. The standardized format aids in quickly identifying the genes associated with the phenotypes and generating scores for the genes. The standardized format also provides flexibility in selecting additional sources for information to use with the clinical decision tool. Another technical advantage of the systems and methods of the present disclosure is portability of the clinical decision tool. The clinical decision tool is easy to distribute to users to deploy locally on a device of a user.

The clinical decision tool facilitates rapid interpretation of clinical whole-genome (WGS) or whole-exome (WES) sequencing results. Both WGS and WES studies typically identify thousands of genetic variants that might be associated with the patient findings, and sifting through the long lists of variants is a manual and time-consuming task that must be performed by a genomics interpretation expert. The systems and methods simplify the manual process by identifying the short list of genes that are most likely associated to the patient's phenotypes, reducing manual review time and expense. The systems and methods provide a lightweight clinical decision tool capable of running locally on a device of a user or accessed through an application programming interface (API) call using the device. One example use of the systems and methods include using the short list of genes in diagnosing a patient. Another example use of the systems and methods include updating a patient's treatment plan using the short list of genes. Another example use of the systems and methods include identifying new associations among genes and phenotypes. Another example use of the systems and methods includes identifying pathogenic variants.

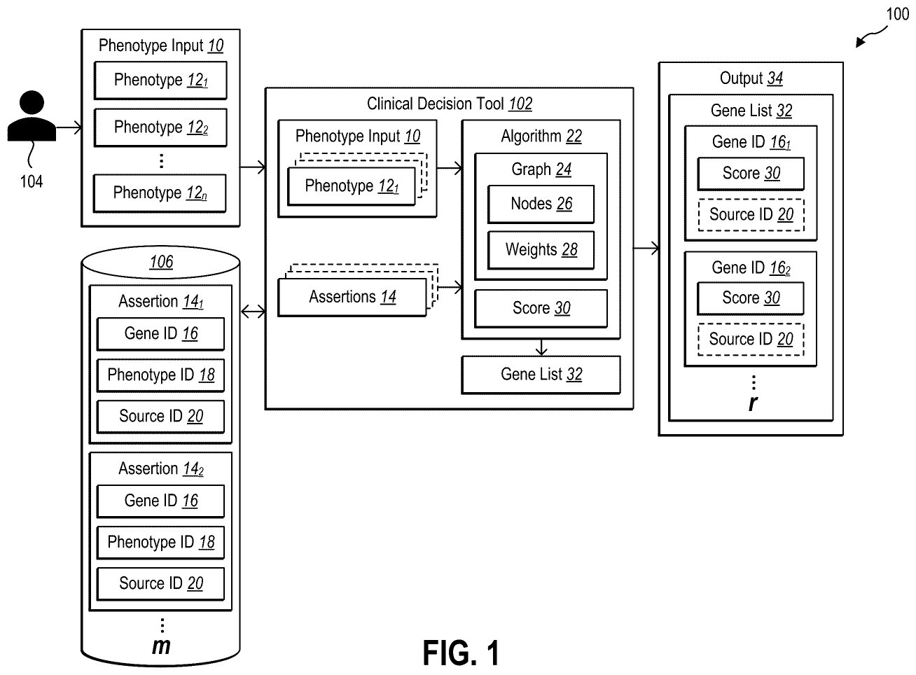

Referring now to FIG. 1, illustrated is an example environment 100 for identifying genes associated with phenotypes. The environment 100 includes a clinical decision tool 102 that receives a phenotype input 10 with a plurality of phenotypes and provides an output 34 with a gene list 32 that includes a plurality of genes associated with the phenotypes inputted.

In some implementations, one or more users 104 provide the phenotype input 10 to the clinical decision tool 102. The users 104 access the clinical decision tool 102 using a computing device. For example, the users 104 access the clinical decision tool 102 using an application on the computing device (e.g., using an API call) or browser on the computing device. In some implementations, the clinical decision tool 102 is local to the computing device of the user 104. In some implementations, the clinical decision tool 102 is on a server (e.g., a cloud server) remote from the computing device of the user 104. In some implementations, the clinical decision tool 102 is hosted on virtual machines in the cloud. In some implementations, the clinical decision tool 102 is on an edge device.

The phenotype input 10 includes a plurality of phenotypes (e.g., phenotype 12₁, phenotype 12₂, phenotype 12₃ up to phenotype 12ₙ, where n is a positive integer). Each phenotype (e.g., the phenotype 12₁, phenotype 12₂, phenotype 12₃) included in the phenotype input 10 describes different symptoms, traits, or characteristics of the individual. For example, the user 104 provides the phenotype input 10 with different phenotypes describing symptoms (e.g., fever, seizures) of an individual with a suspected genetic disorder. In some implementations, the phenotypes (e.g., the phenotype 12₁, phenotype 12₂, phenotype 12₃) include in the phenotype input 10 also have a phenotype ID (e.g., a phenotype ID obtained from the human phenotype ontology (HPO)) that is used to help identify the phenotypes inputted.

The clinical decision tool 102 obtains assertions 14 (e.g., assertion 14₁, assertion 14₂, up to assertion 14ₘ, where m is a positive integer) from a datastore 106. The assertions 14 link a gene to a phenotype. The assertions 14 are in a standard format that contains the gene-to-phenotype associations.

In some implementations, the assertions (e.g., assertion 14₁, assertion 14₂, up to assertion 14ₘ) are obtained from a plurality of sources. Sources include publicly available sources, such as, medical journals and medical publications. Sources may also include private sources, such as, a company's research or university's research. Examples of sources include human phenotype ontology (HPO), human gene mutation database (HGMD), ClinVar, OMIM, OrphaNet, and DeCipher.

In some implementations, the assertions (e.g., assertion 14₁, assertion 14₂) are automatically obtained from information provided by the sources. One example includes a custom parser automatically extracting the assertions from database tables or comma-separated values (CSV) (text) files provided by the sources. The information is automatically analyzed to identify a gene to phenotype association. The assertion is a single piece of evidence extracted from a trusted source of information that links genetic variants in a genomic feature (such as a gene) to a specific phenotype. The assertion is automatically converted into a standard format and saved in the datastore 106. Each assertion includes a gene ID 16 identifying a gene, a phenotype ID 18 identifying a phenotype linked to the gene, and a source ID 20 identifying the source of information used to associate the gene to the phenotype.

In some implementations, the assertions (e.g., assertion 14₁, assertion 14₂) include additional information that aids the users 104 in downstream tasks analyzing the assertions. One example of additional information includes a score indicating a level of confidence of the source in associating the gene to the phenotype. For example, if the medical publication indicated that fifteen different labs identified the gene as linked to the phenotype, the score included with the assertion is higher (e.g., closer to “1”) indicating a high level of confidence the gene is linked to the phenotype. However, if the medical publication indicated labs had conflicting results (some labs identified the link between the gene and the phenotype while other labs identified the gene and phenotype were unrelated), the score included with the assertion is lower (e.g., closer to “0”) indicating a lower level of confidence the gene is linked to the phenotype. Another example of additional information includes age of onset and frequency information. Another example of additional information includes the PubMed IDs from an original source.

The standardized format of the assertions (e.g., assertion 14₁, assertion 14₂) creates an extensible and flexible interface that accommodates multiple sources of information and allows easy addition of new sources of information regarding gene-to-phenotype associations as they arise. The standardized format of the assertions (e.g., assertion 14₁, assertion 14₂) also aids in quickly identifying the genes associated with the phenotypes and generating scores for the genes.

The phenotype input 10 with the plurality of phenotypes (e.g., the phenotype 12₁, phenotype 12₂, phenotype 12₃) and the assertions (e.g., assertion 14₁, assertion 14₂) are provided as input to an algorithm 22. The algorithm 22 scores genes for a relevance to the phenotypes (e.g., the phenotype 12₁, phenotype 12₂, phenotype 12₃) included in the phenotype input 10 and outputs a gene list 32 with genes that are likely to be associated with the phenotypes.

One example of using the clinical decision tool 102 includes the user 104 provides the phenotype input 10 with Telangiectasias as the phenotype 12₁ with a phenotype ID (HP: 000123), runny nose as the phenotype 12₂ with a phenotype ID (HP: 000456), and webbed feet as the phenotype 12₃ with a phenotype ID (HP: 213456). The output 34 provided by the clinical decision tool 102 in response to the algorithm 22 processing the phenotype input 10 and the assertions (e.g., assertion 14₁, assertion 14₂) is the gene list 32. The gene list 32 includes a ranked list of genes that are likely associated with the phenotypes (Telangiectasias, runny nose, and webbed feet). For example, the gene list 32 includes the genes: ENG (the gene ID1 16) with a score 0.987, ACVRL1 (the gene ID2 16) with a score 0.967, SMAD4 (the gene ID3 16) with a score 0.653, and GDF2 (the gene ID4 16) with a score 0.523. The scores provide a level of likelihood that the gene is related to the inputted phenotypes. For example, scores closer to “1” indicate that the gene is likely associated with the phenotypes and scores closer to “0” indicate that the gene is less likely associated with the phenotypes. The user 104 uses in the gene list 32 in identifying gene variations of a patient.

In some implementations, the algorithm 22 is a graph-based algorithm that uses a graph 24 formed using the assertions (e.g., assertion 14₁, assertion 14₂). The nodes 26 in the graph 24 are the phenotypes. Each node has a different phenotype and a list of assertions for the phenotype (e.g., the assertions that include the phenotype ID 18 of the phenotype included in the node). The edges are connections between the nodes 26. An example connection is the same gene (e.g., gene ID 16) is included in the assertions of neighboring nodes 26 in the graph and an edge is provided between the nodes 26 in the graph 24.

Weights 28 are provided to each node 26 in the graph 24 to indicate how close the phenotype of each node 26 is to a phenotype (e.g., the phenotype 12₁, phenotype 12₂, phenotype 12₃) provided in the phenotype input 10. For example, the weights 28 are determined by using random walk traversals of a subset of the nodes in the graph 24 starting from the phenotypes included in the phenotype input 10 and determining how close the nodes are to the phenotypes. The nodes 26 in the graph 24 closer to the phenotypes included in the phenotype input 10 have a higher weight 28 (e.g., closer to “1”) and the nodes 26 in the graph 24 that are further away from the phenotypes included in the phenotype input 10 have a lower weight 28 (e.g., closer to “0”).

The algorithm 22 collects for each node 26 in the graph 24, the assertions (e.g., assertion 14₁, assertion 14₂) and calculates a score 30 for the gene IDs 16 included in the assertions. In some implementations, the algorithm 22 uses a matrix to indicate whether the gene ID 16 is associated with a phenotype (e.g., place a “1” in the matrix if the gene is associated with the phenotype or a “0” in the matrix if the gene is not associated with the phenotype). The algorithm 22 aggregates the scores 30 for the genes across the nodes 26 and applies the weights 28 of the nodes 26 to the scores. An example equation that the algorithm 22 uses in determining the scores 30 is illustrated below in equation (1):

$$1 - {e}^{- 0.5 \sum {a}_{i}} \qquad ( 1 )$$

where aᵢ is a score from the assertion i weighted by the node value (e.g., the weight 28).

The algorithm 22 outputs the genes list 32 with a list of genes (e.g., the gene ID1 16, the gene ID2 16, the gene ID3 16). In some implementations, the gene list 32 includes the list of genes in a ranked order using the scores 30 of the genes where the genes are placed in descending order by the score 30 with a gene with the highest score 30 placed at the top of the gene list 32. In some implementations, the gene list 32 includes the score 30 for each of the genes. For example, the score 30 is an overall gene relevance score the input phenotypes (e.g., the phenotype 12₁, phenotype 12₂, phenotype 12₃). Another example includes the score 30 is an individual score for a relevance of a gene to each input phenotype.

The clinical decision tool 102 provides an output 34 with the gene list 32 in response to the phenotype input 10. In some implementations, the output 34 is presented on a graphical user interface of the device of the user 104. The output 34 includes the gene list 32 with the genes identified by the gene IDs (e.g., the gene ID1 16, the gene ID2 16, the gene ID3 16) and the score 30 indicating a relevance of the genes to the input phenotypes (e.g., the phenotype 12₁, phenotype 12₂, phenotype 12₃).

In some implementations, the gene list 32 includes the source ID 20 that provided the assertions (e.g., the (e.g., assertion 14₁, assertion 14₂) used in determining whether the genes were related to the input phenotypes. In some implementations, the gene list 32 includes a link to the source ID 20 that provides access to the original source where the information was gathered for determining that the gene is related to the phenotype. For example, if the user 104 clicks or otherwise selects the link, the original source is presented nearby the output 34.

The output 34 identifies a short list of genes (e.g., a subset of the genes with a score 30 above a threshold level) that are most likely to be associated to an individual's phenotypes. The users 104 may use the output 34 to aid in discovering different genes associated with phenotypes. For example, the users 104 may provide different phenotype inputs 10 to the clinical decision tool 102 and use the output 34 to identify different genes associated with the phenotype input 10. The users 104 may also use the output 34 to find pathogenic variants faster.

In some implementations, one or more computing devices (e.g., servers and/or devices) are used to perform the processing of the environments 100. The one or more computing devices may include, but are not limited to, server devices, cloud virtual machines, personal computers, a mobile device, such as, a mobile telephone, a smartphone, a PDA, a tablet, or a laptop, and/or a non-mobile device. The features and functionalities discussed herein in connection with the various systems may be implemented on one computing device or across multiple computing devices. For example, the clinical decision tool 102 and the datastore 106 is implemented wholly on a computing device. Another example includes one or more subcomponents of the clinical decision tool 102 and/or the datastore 106 implemented across multiple computing devices. Moreover, in some implementations, one or more subcomponent of the clinical decision tool 102 and/or the datastore 106 may be implemented are processed on different server devices of the same or different cloud computing networks.

In some implementations, each of the components of the environment 100 is in communication with each other using any suitable communication technologies. In addition, while the components of the environment 100 are shown to be separate, any of the components or subcomponents may be combined into fewer components, such as into a single component, or divided into more components as may serve a particular implementation. In some implementations, the components of the environments 100 include hardware, software, or both. For example, the components of the environment 100 may include one or more instructions stored on a computer-readable storage medium and executable by processors of one or more computing devices. When executed by the one or more processors, the computer-executable instructions of one or more computing devices can perform one or more methods described herein. In some implementations, the components of the environment 100 include hardware, such as a special purpose processing device to perform a certain function or group of functions. In some implementations, the components of the environment 100 include a combination of computer-executable instructions and hardware.

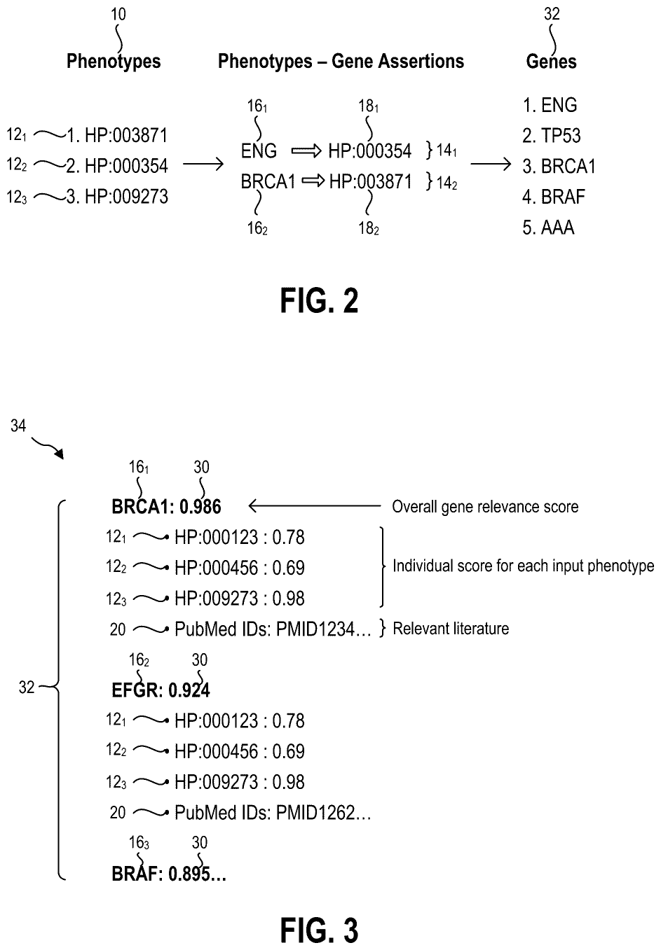

FIG. 2 illustrates an example phenotype to gene assertions 14₁, 14₂ for use with the clinical decision tool 102 (FIG. 1). In the illustrated example, the phenotype input 10 includes the phenotype 12₁ (HP: 003871), the phenotype 12₂ (HP: 000354), and the phenotype 12₃ (HP: 009273). The assertion 14₁ links the gene ID 16₁ (ENG) to the phenotype ID 18₁ (HP: 000354) and the assertion 14₂ links the gene ID 16₂ (BRCA1) to the phenotype ID 18₂ (HP: 003871). The algorithm 22 (FIG. 1) receives the phenotype input 10 and the assertions (e.g., the assertions 14₁, 14₂) obtained from the datastore 106 (FIG. 1) and uses the assertions 14₁, 14₂ to identify a gene list 32 with a list of genes that are associated with the phenotype input 10. For example, the gene list 32 includes five genes (ENG, TP53, BRCA1, BRAF, AAA) that the algorithm 22 identified as associated with the phenotype input 10. In some implementations, the genes included in the gene list 32 are placed in a ranked order based on an overall score (e.g., the score 30) indicating a relevance of the gene to the phenotype input 10.

FIG. 3 illustrates an example output 34 of the clinical decision tool 102 (FIG. 1). The output 34 includes a gene list 32 with a plurality of gene IDs 16₁, 16₂, 16₃ that the algorithm 22 (FIG. 1) identified as being related to the phenotypes (the phenotype 12₁, the phenotype 12₂, the phenotype 12₂) included in the phenotype input 10 (FIG. 1) provided to the clinical decision tool 102. The output 34 also includes a score 30 for each gene ID and the source ID 20 that provided the assertions (e.g., the assertions 14₁, 14₂ (FIG. 1)) used by the algorithm 22 in determining whether the genes are related to the phenotypes. For example, the gene ID 16₁ (BRCA1) has an overall gene relevance score of 0.986 to the phenotypes included in the phenotype input 10. The gene ID 16₂ (EGFR) has an overall gene relevance score of 0.924 to the phenotypes included in the phenotype input 10. The output 34 also includes individual scores of relevance of the gene to each individual phenotype (the phenotype 12₁, the phenotype 12₂, the phenotype 12₂) included in the phenotype input 10 (FIG. 1). In some implementations, the output 34 is presented on a user interface of a device of the user 104.

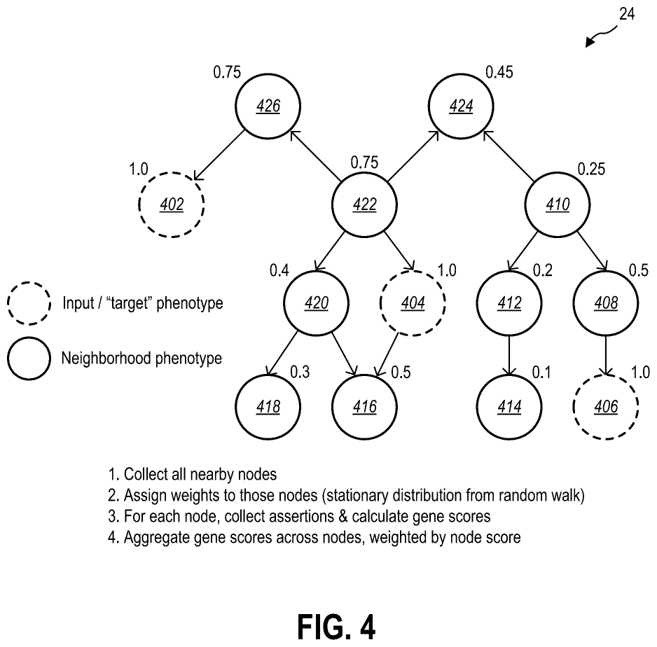

FIG. 4 illustrates an example graph 24 for use with an algorithm 22 (FIG. 1) to identify genes associated with phenotype input 10 (FIG. 1). The graph 24 includes a plurality of nodes (e.g., the nodes 26 (FIG. 1), where each node is a phenotype. In some implementations, an edge between nodes of the graph 24 indicates a relationship between the nodes. Each node has a plurality of assertions (e.g., the assertions 14₁, 14₂ (FIG. 1)) for the phenotype. The graph 24 includes input nodes 402, 404, 406 that correspond three phenotypes (e.g., the phenotype 12₁, the phenotype 12₂, the phenotype 12₂) provided in the phenotype input 10. The graph 24 also includes a plurality of neighbor nodes 408, 410, 412, 414, 416, 418, 420, 422, 424, 426 in a vicinity of the input nodes 402, 404, 406.

The algorithm 22 collects nearby nodes (e.g., the neighbor nodes) of the input nodes 402, 404, 406 in the graph 24 and assigns weights 28 (FIG. 1) to the nearby nodes. In some implementations, the weight 28 identifies a level of similarity among the phenotypes in the nearby nodes. In some implementations, the algorithm 22 performs random walks of the nodes of the graph 24 starting from the different input nodes 402, 404, 406. For each node in the graph 24, the algorithm 22 collects assertions and calculates a gene score for the genes included in the assertions.

For example, the algorithm 22 computes for each gene a gene score indicating a frequency of each gene in the assertions of the node. The algorithm 22 aggregates the gene score across the nodes in the graph 24, weighted by the node score. The algorithm uses the aggregated gene score across the nodes in the graph 24 in determining the genes list 32 (FIG. 1) with the list of genes that are associated with the phenotype input 10.

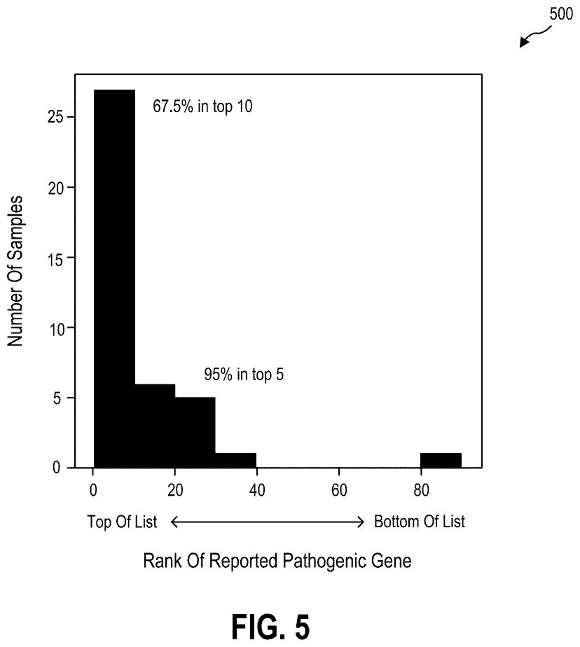

FIG. 5 illustrates an example graph 500 with reported pathogenic genes from clinical exomes probands using the clinical decision tool 102 (FIG. 1). For example, 40 exome probands with pathogenic variants were provided to the clinical decision tool 102.

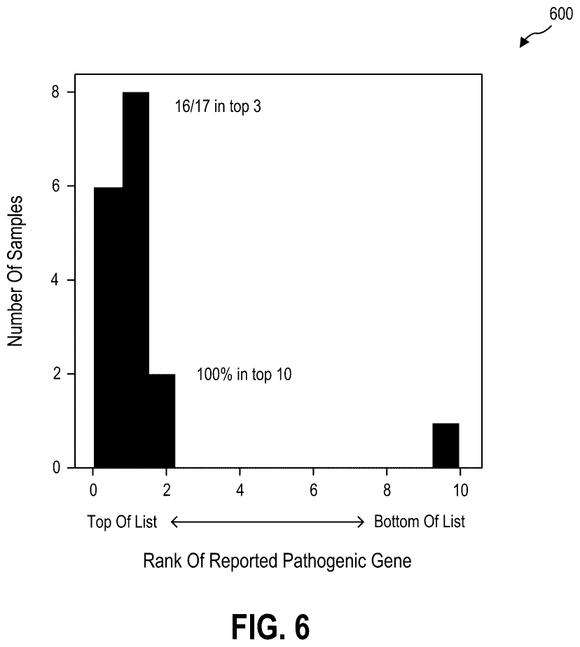

FIG. 6 illustrates an example graph 600 with reported pathogenic genes from clinical exome trios using the clinical decision tool 102 (FIG. 1). For example, 17 exome trios with pathogenic variants were provided to the clinical decision tool 102.

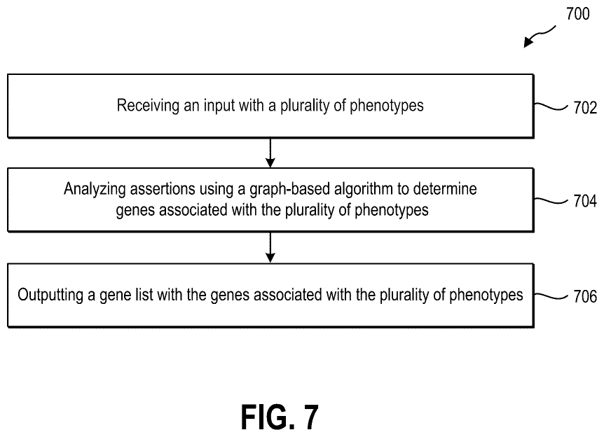

FIG. 7 illustrates an example method 700 of identifying genes associated with phenotypes. The actions of the method 700 are discussed below in reference to FIGS. 1-4.

At 702, the method 700 includes receiving an input with a plurality of phenotypes. The clinical decision tool 102 receives an input (e.g., the phenotype input 10) with a plurality or phenotypes (e.g., the phenotype 12₁, the phenotype 12₂, the phenotype 12₂).

At 704, the method 700 includes analyzing assertions using a graph-based algorithm to determine genes associated with the plurality of phenotypes. The clinical decision tool 102 determines genes (e.g., the gene list 32) associated with the plurality of phenotypes (e.g., the phenotype 12₁, the phenotype 12₂, the phenotype 12₂) by analyzing the assertions (e.g., the assertions 14₁, 14₂) using a graph-based algorithm (e.g., the algorithm 22). In some implementations, each assertion is in a standard format that associates a gene with a phenotype.

In some implementations, each assertion further includes a gene identification (ID) for the gene, a human phenotype ontology (HPO) identification (ID) for the phenotype, and a source identification (ID) for an original source that provided information for associating the gene to the phenotype. In some implementations, a link is output that provides access to a source used in determining that the genes are associated with the plurality of phenotypes. For example, if the user 104 selects the link, the original source is presented on a display of a device. In some implementations, each assertion further includes a score indicating a level of confidence that the gene is associated with the phenotype. In some implementations, each assertion further includes age of onset information or frequency information.

In some implementations, a datastore (e.g., the datastore 106) is accessed of the assertions. For example, the assertions are automatically added to the datastore in the standard format from a plurality of sources in response to automatically obtaining the information from the plurality of sources. In some implementations, a plurality of sources are accessed for the assertions, the assertions are automatically converted into a standard format, and the assertions are stored in the datastore in the standard format.

In some implementations, the graph-based algorithm uses a graph (e.g., the graph 24) that includes a plurality of nodes (e.g., the nodes 26), where each node is a different phenotype and includes a plurality of assertions associated with the phenotype. In some implementations, the graph-based algorithm identifies a node corresponding to a phenotype of the plurality of phenotypes; collects nearby nodes in the graph of the phenotype; assigs weights to the nearby nodes by performing a random graph walk; for each node, collects the plurality of assertions for the phenotype and calculates gene scores; and aggregates the gene scores across the nearby nodes.

At 706, the method 700 includes outputting a gene list with the genes associated with the plurality of phenotypes. The clinical decision tool 102 outputs a gene list 32 with the genes (e.g., the gene IDs 16₁, 16₂, 16₃) associated with the plurality of phenotypes (e.g., the phenotype 12₁, the phenotype 12₂, the phenotype 12₂). In some implementations, the gene list 32 is presented on a display of a device of the user 104. In some implementations, the user 104 identifies pathogenic variants in an individual using the gene list 32. In some implementations, the user 104 identifies genes associated with a disease in a patient using the gene list 32. In some implementations, the user 104 uses the gene list 32 in narrowing down the genes to analyze further. In some implementations, the user 104 uses the gene list 32 in isolating genes to use in diagnosing a disease in a patient. In some implementations, the user 104 uses the gene list 32 in confirming a diagnosis of a disease in a patient. In some implementations, the user 104 updates a variant classification system using the information in the gene list 32.

In some implementations, a message (e.g., an email message) is sent with the gene list 32 with the genes associated with the plurality of phenotypes. For example, the message is sent to a plurality of users notifying the users of the genes associated with the plurality of phenotypes. One example is the message with the gene list 32 is sent to variant scientists. The message includes new associations of the gene IDs with the plurality of phenotypes (e.g., the associations were not previously known) and the variant scientists update a variant classification system with the new associations identified in the gene list 32.

In some implementations, the gene scores are used to determine the genes associated with the plurality of phenotypes. In some implementations, the genes associated with the plurality of phenotypes are ranked based on the scores and the genes are outputted in the gene list 32 in a ranked order. The genes with a higher ranking are outputted first relative to the genes with a lower ranking. In some implementations, the user 104 uses the gene list 32 in sorting variants found in an individual.

The method 700 is used to automatically identify genes that have a clinical association with one or more phenotypes.

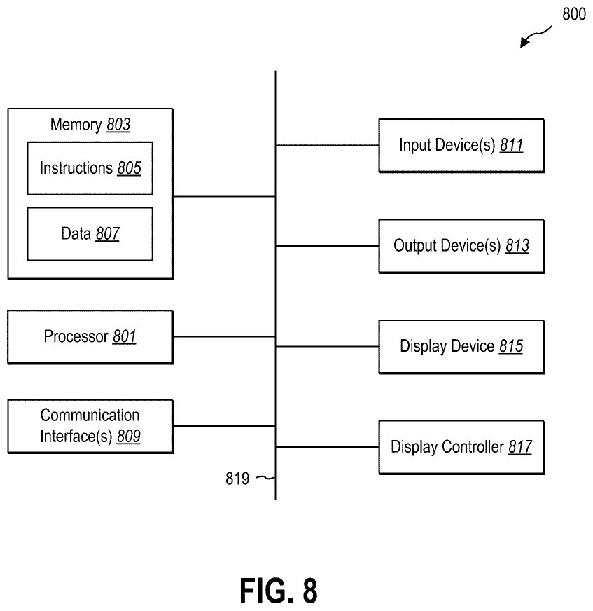

FIG. 8 illustrates components that may be included within a computer system 800. One or more computer systems 800 may be used to implement the various methods, devices, components, and/or systems described herein.

The computer system 800 includes a processor 801. The processor 801 may be a general-purpose single or multi-chip microprocessor (e.g., an Advanced RISC (Reduced Instruction Set Computer) Machine (ARM)), a special purpose microprocessor (e.g., a digital signal processor (DSP)), a graphics processing unit (GPU), a microcontroller, a programmable gate array, etc. The processor 801 may be referred to as a central processing unit (CPU). Although just a single processor 801 is shown in the computer system 800 of FIG. 8, in an alternative configuration, a combination of processors (e.g., an ARM and DSP) could be used.

The computer system 800 also includes memory 803 in electronic communication with the processor 801. The memory 803 may be any electronic component capable of storing electronic information. For example, the memory 803 may be embodied as random access memory (RAM), read-only memory (ROM), magnetic disk storage mediums, optical storage mediums, flash memory devices in RAM, on-board memory included with the processor, erasable programmable read-only memory (EPROM), electrically erasable programmable read-only memory (EEPROM) memory, registers, and so forth, including combinations thereof.

Instructions 805 and data 807 may be stored in the memory 803. The instructions 805 may be executable by the processor 801 to implement some or all of the functionality disclosed herein. Executing the instructions 805 may involve the use of the data 807 that is stored in the memory 803. Any of the various examples of modules and components described herein may be implemented, partially or wholly, as instructions 805 stored in memory 803 and executed by the processor 801. Any of the various examples of data described herein may be among the data 807 that is stored in memory 803 and used during execution of the instructions 805 by the processor 801.

A computer system 800 may also include one or more communication interfaces 809 for communicating with other electronic devices. The communication interface(s) 809 may be based on wired communication technology, wireless communication technology, or both. Some examples of communication interfaces 809 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system 800 may also include one or more input devices 811 and one or more output devices 813. Some examples of input devices 811 include a keyboard, mouse, microphone, remote control device, button, joystick, trackball, touchpad, and lightpen. Some examples of output devices 813 include a speaker and a printer. One specific type of output device that is typically included in a computer system 800 is a display device 815. Display devices 815 used with embodiments disclosed herein may utilize any suitable image projection technology, such as liquid crystal display (LCD), light-emitting diode (LED), gas plasma, electroluminescence, or the like. A display controller 817 may also be provided, for converting data 807 stored in the memory 803 into text, graphics, and/or moving images (as appropriate) shown on the display device 815.

The various components of the computer system 800 may be coupled together by one or more buses, which may include a power bus, a control signal bus, a status signal bus, a data bus, etc. For the sake of clarity, the various buses are illustrated in FIG. 8 as a bus system 819.

In some implementations, the various components of the computer system 800 are implemented as one device. For example, the various components of the computer system 800 are implemented in a mobile phone or tablet. Another example includes the various components of the computer system 800 implemented in a personal computer. Another example includes the various components of the computer system 800 implemented in the cloud. Another example includes the various components of the computer system 800 implemented on an edge device.

As illustrated in the foregoing discussion, the present disclosure utilizes a variety of terms to describe features and advantages of the model evaluation system. Additional detail is now provided regarding the meaning of such terms. For example, as used herein, a “machine learning model” refers to a computer algorithm or model (e.g., a classification model, a clustering model, a regression model, a language model, an object detection model, a probabilistic graphical model) that can be tuned (e.g., trained) based on training input to approximate unknown functions. For example, a machine learning model may refer to a neural network (e.g., a convolutional neural network (CNN), deep neural network (DNN), recurrent neural network (RNN)), or other machine learning algorithm or architecture that learns and approximates complex functions and generates outputs based on a plurality of inputs provided to the machine learning model. As used herein, a “machine learning system” may refer to one or multiple machine learning models that cooperatively generate one or more outputs based on corresponding inputs. For example, a machine learning system may refer to any system architecture having multiple discrete machine learning components that consider different kinds of information or inputs.

The techniques described herein may be implemented in hardware, software, firmware, or any combination thereof, unless specifically described as being implemented in a specific manner. Any features described as modules, components, or the like may also be implemented together in an integrated logic device or separately as discrete but interoperable logic devices. If implemented in software, the techniques may be realized at least in part by a non-transitory processor-readable storage medium comprising instructions that, when executed by at least one processor, perform one or more of the methods described herein. The instructions may be organized into routines, programs, objects, components, data structures, etc., which may perform particular tasks and/or implement particular data types, and which may be combined or distributed as desired in various implementations.

Computer-readable mediums may be any available media that can be accessed by a general purpose or special purpose computer system. Computer-readable mediums that store computer-executable instructions are non-transitory computer-readable storage media (devices). Computer-readable mediums that carry computer-executable instructions are transmission media. Thus, by way of example, and not limitation, implementations of the disclosure can comprise at least two distinctly different kinds of computer-readable mediums: non-transitory computer-readable storage media (devices) and transmission media.

As used herein, non-transitory computer-readable storage mediums (devices) may include RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

The steps and/or actions of the methods described herein may be interchanged with one another without departing from the scope of the claims. In other words, unless a specific order of steps or actions is required for proper operation of the method that is being described, the order and/or use of specific steps and/or actions may be modified without departing from the scope of the claims.

The term “determining” encompasses a wide variety of actions and, therefore, “determining” can include calculating, computing, processing, deriving, investigating, looking up (e.g., looking up in a table, a database, a datastore, or another data structure), ascertaining and the like. Also, “determining” can include receiving (e.g., receiving information), accessing (e.g., accessing data in a memory) and the like. Also, “determining” can include resolving, selecting, choosing, establishing, predicting, inferring, and the like.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one implementation” or “an implementation” of the present disclosure are not intended to be interpreted as excluding the existence of additional implementations that also incorporate the recited features. For example, any element described in relation to an implementation herein may be combinable with any element of any other implementation described herein. Numbers, percentages, ratios, or other values stated herein are intended to include that value, and also other values that are “about” or “approximately” the stated value, as would be appreciated by one of ordinary skill in the art encompassed by implementations of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to implementations disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the implementations that falls within the meaning and scope of the claims is to be embraced by the claims.

The present disclosure may be embodied in other specific forms without departing from its spirit or characteristics. The described implementations are to be considered as illustrative and not restrictive. The scope of the disclosure is, therefore, indicated by the appended claims rather than by the foregoing description. Changes that come within the meaning and range of equivalency of the claims are to be embraced within their scope.

1. A method, comprising:
receiving an input with a plurality of phenotypes;
analyzing assertions using a graph-based algorithm to determine genes associated with the plurality of phenotypes, wherein each assertion is in a standard format that associates a gene with a phenotype; and
outputting a gene list with the genes associated with the plurality of phenotypes.

2. The method of claim 1, wherein each assertion further includes a gene identification (ID) for the gene, a human phenotype ontology (HPO) identification (ID) for the phenotype, and a source identification (ID) for a source that provided information for associating the gene to the phenotype.

3. The method of claim 2, further comprising:
outputting a link that provides access to the source.

4. The method of claim 1, wherein each assertion further includes a score indicating a level of confidence that the gene is associated with the phenotype.

5. The method of claim 1, wherein each assertion further includes age of onset information or frequency information.

6. The method of claim 1, further comprising:
accessing a datastore of the assertions, wherein the assertions are automatically added to the datastore in the standard format from a plurality of sources.

7. The method of claim 1, further comprising:
accessing a plurality of sources for the assertions;
converting the assertions into the standard format; and
storing the assertions in a datastore.

8. The method of claim 1, further comprising:
ranking the genes associated with the plurality of phenotypes; and
outputting the genes in the gene list in response to the ranking, wherein the genes with a higher ranking are outputted first relative to the genes with a lower ranking.

9. The method of claim 1, wherein the graph-based algorithm uses a graph that includes a plurality of nodes, where each node is a different phenotype and includes a plurality of assertions associated with the phenotype.

10. The method of claim 9, wherein the graph-based algorithm further includes:
identifying a node corresponding to a phenotype of the plurality of phenotypes;
collecting nearby nodes in the graph of the phenotype;
assigning weights to the nearby nodes by performing a random graph walk;
for each node, collecting the plurality of assertions for the phenotype and calculating gene scores; and
aggregating the gene scores across the nearby nodes.

11. The method of claim 10, further comprising:
using the gene scores to determine the genes associated with the plurality of phenotypes; and
outputting the gene list with the genes in a ranked order based on the gene scores.

12. A system, comprising:
a memory to store data and instructions; and
a processor operable to communicate with the memory, wherein the processor is operable to:
receive an input with a plurality of phenotypes;
analyze assertions using a graph-based algorithm to determine genes associated with the plurality of phenotypes, wherein each assertion is in a standard format that associates a gene with a phenotype; and
output a gene list with the genes associated with the plurality of phenotypes.

13. The system of claim 12, wherein each assertion further includes a gene identification (ID) for the gene, a human phenotype ontology (HPO) identification (ID) for the phenotype, and a source identification (ID) for a source that provided information for associating the gene to the phenotype.

14. The system of claim 12, wherein each assertion further includes a score indicating a level of confidence that the gene is associated with the phenotype, age of onset information, and frequency information.

15. The system of claim 12, wherein the processor is further operable to access a datastore of the assertions, wherein the assertions are automatically added to the datastore in the standard format from a plurality of sources.

16. The system of claim 12, wherein the processor is further operable to:
access a plurality of sources for the assertions;
convert the assertions into the standard format; and
store the assertions in a datastore.

17. The system of claim 12, wherein the processor is further operable to:
rank the genes associated with the plurality of phenotypes; and
output the genes in the gene list in response to the ranking, wherein the genes with a higher ranking are outputted first relative to the genes with a lower ranking.

18. The system of claim 12, wherein the graph-based algorithm uses a graph that includes a plurality of nodes, where each node is a different phenotype and includes a plurality of assertions associated with the phenotype.

19. The system of claim 18, wherein the processor is further operable to:
identify a node corresponding to a phenotype of the plurality of phenotypes;
collect nearby nodes in the graph of the phenotype;
assign weights to the nearby nodes by performing a random graph walk;
for each node, collecting the plurality of assertions for the phenotype and calculating gene scores; and
aggregate the gene scores across the nearby nodes.

20. The system of claim 19, wherein the processor is further operable to:
use the gene scores to determine the genes associated with the plurality of phenotypes; and
output the gene list with the genes in a ranked order based on the gene scores.
